(19) United States
(12) Patent Application Publication
(10) Pub. No.: US 2002/0041823 A1
(43) Pub. Date: Apr. 11, 2002

(54) Storage tank for a gaseous medium

(76) Inventor: Janusz Blaszczyk, Richmond (CA)

Correspondence Address: Evenson, McKeown,, Edwards & Lenahan, P.L.L.C., Suite 700, 1200 G Street, N.W., Washington, DC, 20005, US

(21) Appl. No.: 09/924,175

(22) Filed: Aug. 8, 2001

(30) Foreign Application Priority Data
Aug. 8, 2000 (EP) 001 16 975.4

(51) Int. Cl. A01N 1/00; A61L 2/00; A61L 9/00; C23F 11/00; A61L 11/00

(52) U.S. Cl. 422/1

(57) ABSTRACT

A storage tank for a medium has a first volume provided for containing the gaseous medium and a second volume provided for at least receiving the liquid medium. A heater for heating the medium and inlet and outlet means for the medium are provided. The first volume is inside a first tank and the second volume is inside a second tank. The second tank is in fluid connection with said first tank via at least one fluid conduit and said second vessel is surrounded by said first vessel. The storage tank can store hydrogen for a fuel cell operated vehicle.

### BACKGROUND AND SUMMARY OF THE INVENTION

This application claims the priority of German Patent Document 001 16 975.4, filed Aug. 8, 2000, the disclosure of which is expressly incorporated by reference herein.

The invention relates to a storage tank for a gaseous medium. Preferred embodiments relate to a storage tank for a medium with a first volume provided for containing a gaseous phase of said medium and a second volume provided for containing a liquid phase of said medium, a heater for heating said medium, an inlet and an outlet for said medium.

German Patent Document DE 42 12 626 A1 discloses a storage tank for liquid hydrogen. A pump mounted inside the tank extends from the liquid containing volume to the hydrogen gas containing volume of the tank. An evaporation heater is provided in the gas containing volume or inside the pump. Liquid hydrogen is pumped into the gas containing volume and is evaporated inside the pump or the gas room until the hydrogen gas pressure reaches a nominal pressure value, e.g. 5 bar. At pressure values above the nominal pressure value the pump stops until the pressure drops under the nominal value. In order to prevent slopping and mixing of gaseous and liquid hydrogen due to rapid movements of the tank, e.g., when mounted in a vehicle, several metal sheets are arranged vertically and parallel to each other.

An object of the invention is to specify a storage tank for a medium which is capable of storing a gaseous medium and where simpler filling stations can be used.

This object is achieved according to preferred embodiments of the invention by providing a storage tank for a medium with a first volume provided for containing a gaseous phase of said medium and a second volume provided for containing a liquid phase of said medium, a heater for heating said medium, an inlet and an outlet for said medium, wherein the first volume is inside a first vessel and the second volume is inside a second vessel, wherein said second vessel is in fluid connection with said first vessel via at least one fluid conduit, and wherein said second vessel is surrounded by said first vessel.

The storage tank according to the invention comprises a storage tank for a gaseous high pressure medium in fluid connection with an evaporating tank for a liquid phase of said medium wherein a respective first inlet and outlet is provided for filling or discharging said gaseous medium into or out of said first tank and a respective second inlet and outlet is provided for filling or discharging said liquid medium into or out of said second tank.

The storage tank according to the invention combines the simplicity of a storage system for a high pressure compressed gaseous medium and the advantages of a cryogenic liquid medium filling method at more or less ambient pressure. The invention is especially advantageous for hydrogen.

In a preferred embodiment the tank is part of a motor vehicle propelled by a hydrogen combustion engine or a fuel cell system.

Another advantage of preferred embodiments of the invention resides in the fact that the tank can be filled either with cryogenic liquid, preferably at ambient or moderate pressure, or with gaseous medium, preferably at high pressure.

It is favorable that, if part of a vehicle, the vehicle can provide one tank either for gaseous or for liquid fuel. Therefore, the use of such a vehicle is not restricted to the access to a specified kind of filling stations.

Further advantages and refinements of the invention emerge from the rest of the claims and the description.

Other objects, advantages and novel features of the present invention will become apparent from the following detailed description of the invention when considered in conjunction with the accompanying drawings.

### BRIEF DESCRIPTION OF THE DRAWINGS

FIG. 1 shows a first preferred embodiment of a storage tank according to the invention with two containers for a gaseous and a liquid medium; and

FIG. 2 shows a cross section of said first preferred embodiment of a storage tank according to the invention.

### DETAILED DESCRIPTION OF THE DRAWINGS

The invention can be advantageously used in motor vehicles where hydrogen is used as a fuel in a combustion motor or in fuel cell vehicles as fuel for a fuel cell system but is not restricted to an automotive application. The tank is also suitable for low pressure cryogenic filling and high pressure storage of other cryogenic media than hydrogen.

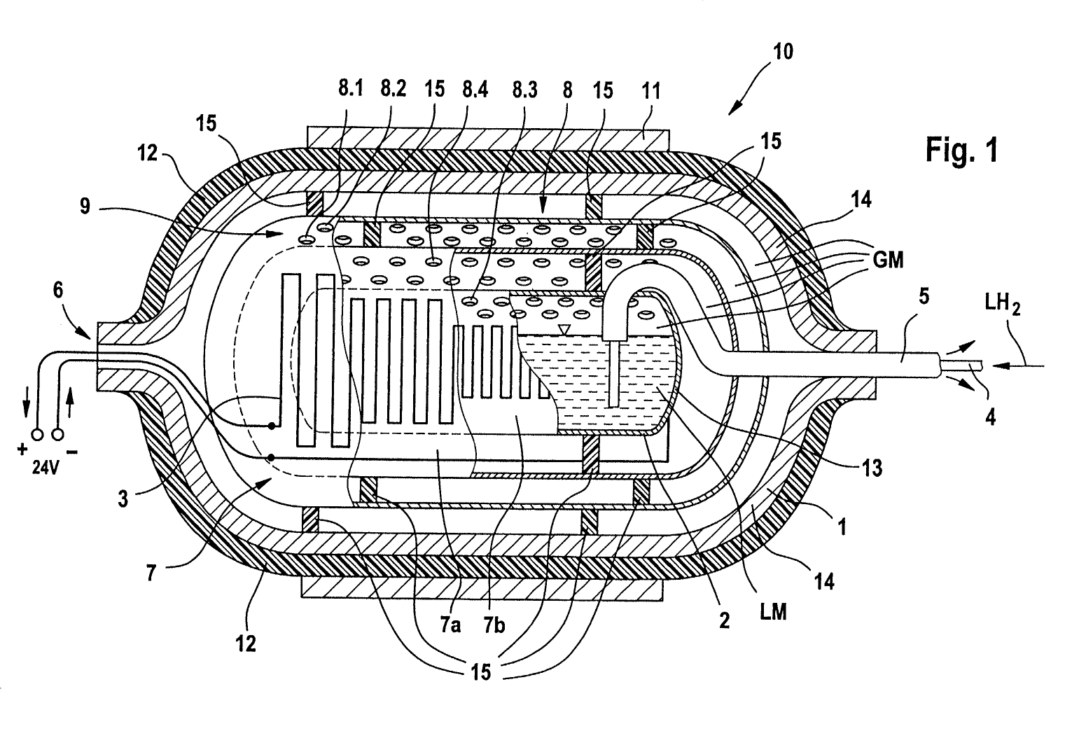

FIG. 1 illustrates a preferred hydrogen tank system.

The tank 10 comprises a first vessel 1 with a first vessel wall 14 for compressed gaseous hydrogen GM. The gaseous hydrogen is stored at high pressures of at least 100 bar, preferably more than 300 bar, and a second tank 2 for liquid hydrogen LM. The outer wall 14 of the first tank 1 is preferably at ambient temperature whereas the outer wall 13 of the second vessel 2 is preferably at liquid hydrogen temperatures.

The tank 10 has preferably two fueling inlets, one inlet/outlet 6 for compressed hydrogen GM connected to the first vessel 1 and one inlet/outlet 4, 5 in the first vessel 1 for liquid hydrogen LM connected with the second vessel 2. The inlet/outlet 4, 5 and 6 can be arranged on opposite sides of the tank 10 or on the same side of the tank 10 or perpendicular to each other. The inlet/outlet 4, 5 for the liquid medium LM comprises preferably two concentric pipes with an inlet pipe 4 and an outlet pipe 5 (or vice versa).

The liquid hydrogen inlet pipe 4 feeds liquified gas at around −250° C. and at relatively low pressures of 3 to 6 bar to the second vessel 2. The second vessel 2 is preferably a thin steel wall container. Alternatively aluminum or carbon fiber material can also be used. The second vessel 2 is preferably located inside the first tank 1 which is the compressed hydrogen tank. The outer wall 13 of the second vessel 2 is thermally isolated from the outer wall 14 of the first tank 1. After the liquid totally evaporates, both vessels 1, 2 contain the high pressure compressed gaseous medium.

The second vessel 2 has at least one fluid conduit 8 to release hydrogen gas evaporated from the liquid from the second vessel 2 into the first vessel 1. The fluid conduit 8 can be a syphon-like outlet to transfer the liquid medium from the second vessel 2 into the first vessel 1.

In a preferred embodiment of the invention the outer wall 13 of the second vessel 2 has openings 8.1, 8.2, 8.3, 8.4 in its top region 9 to allow evaporated hydrogen to fill the first vessel 1. The second vessel 2 is surrounded with several thermal screen layers 7a, 7b, preferably metal shields, in order to slow down the process of liquid hydrogen evaporation and to protect the first vessel wall 14 from being exposed to the extreme temperature difference between inner and outer vessel surface, which would be dangerous for the reinforcement wrapping material 12 which surrounds the outer wall 14 of the first vessel 1.

The perforation holes 8.1, 8.2, 8.3, 8.4 in the thermal screen layers should he staggered so that the thermal radiation shields 7a, 7b, are optically tight so that heating of the liquid hydrogen by direct thermal radiation from the outer walls 14 of vessel 1 which are at ambient temperature can be avoided.

The thermal screen layers can be made of any reflective material such as Mylar foil (polyethylene terephthalate) with reflective coating.

Mountings 15 are located between the first and second vessel walls 13, 14 and between the thermal screen layers 7a, 7b, in order to stabilize the spacing between the components. Preferably the mountings 15 are made of thermally insulating material and/or honeycomb structure.

While filling the second vessel 2 with liquid hydrogen the liquid hydrogen outlet pipe 5 can provide a signal that the required volume of liquid hydrogen LM has been delivered. Alternatively a sensor responsive to the filling level can be located in the second vessel 2. The amount of liquid hydrogen LM corresponds to the amount of hydrogen which expands in the volume of first vessel 1 and second vessel 2 to working pressure after the system temperature stabilizes.

The thermal screen layers 7a, 7b, reduce the convection and radiation heat exchange rates between inner walls of the first vessel 1 and outer walls of the second vessel 2. The perforation holes 8.1, 8.2, 8.3, 8.4 in the thermal screen layers and in the wall of the second tank 2 allow sufficient gas expansion.

The total volume provided for containing said gaseous medium GM in the first vessel 1 including space between the screen 7a, 7b, and in the second vessel 2 is at least equal to the volume of the hydrogen necessary to contain the completely evaporated liquid hydrogen LM at a working pressure p_{w} of the gaseous medium.

The working pressure p_{w} for the first vessel 1 is well above 100 bar. The heater 3 evaporates all hydrogen from the second vessel 2 so that the pressure p_{w} can be established within the volume of the tank 10. Such a pressure can be several hundred bar, e.g., 350 up to 700 bar. Additionally the heater 3 can provide enough energy to keep the temperature of the wall 14 of the first vessel 1 well below a critical temperature in order to avoid damage due to exposure of the wall 14 to low temperatures.

To stabilize the temperature of the storage tank 10 an additional heater 11 can be located around the outer wall 14 of the first vessel 1. The heater 11 can also be used to increase the working pressure level in the tank 10 by heating up the wall 14 of the first vessel 1.

To support the evaporation of liquid hydrogen a heating means 3 is in thermal contact with the screens 7a, 7b and/or the outer wall 13 of the second vessel 2. The heating means 3 can be an electric heater heating an electric resistance via an electric current or another type of heater. The heater 3 can be provided on each single shield 7a, 7b, of the screen means 7 or only on one or selected screens 7a, 7b, of the screen means 7. The second vessel 2 receiving the liquid medium LM is not used to store the cryogenic medium LM but to serve as a medium reservoir for the high-pressure gaseous medium GM. Preferably the liquid medium LM is evaporated shortly after being filled into the second vessel 2. The density of the medium, e.g. hydrogen, in the liquid phase is much higher than in the gas phase. Therefore, a large amount of medium can be transferred into the tank 10 in the liquid phase.

The heating means 3 and the additional heater 11 can be used together or separately based on thermal calculations of the given tank system. The number of thermal screens 7a, 7b, will depend on the design of the first vessel 1 or in other words on the acceptable temperature difference between the outer wall of the first vessel 1 and the outer surface of the reinforcement means 12.

Preferably the reinforcement material is carbon fiber. Alternatively the reinforcement material comprises steel.

The thermal screen layers 7a, 7b, and/or the mountings 15 can be alternatively replaced by a honeycomb structure.

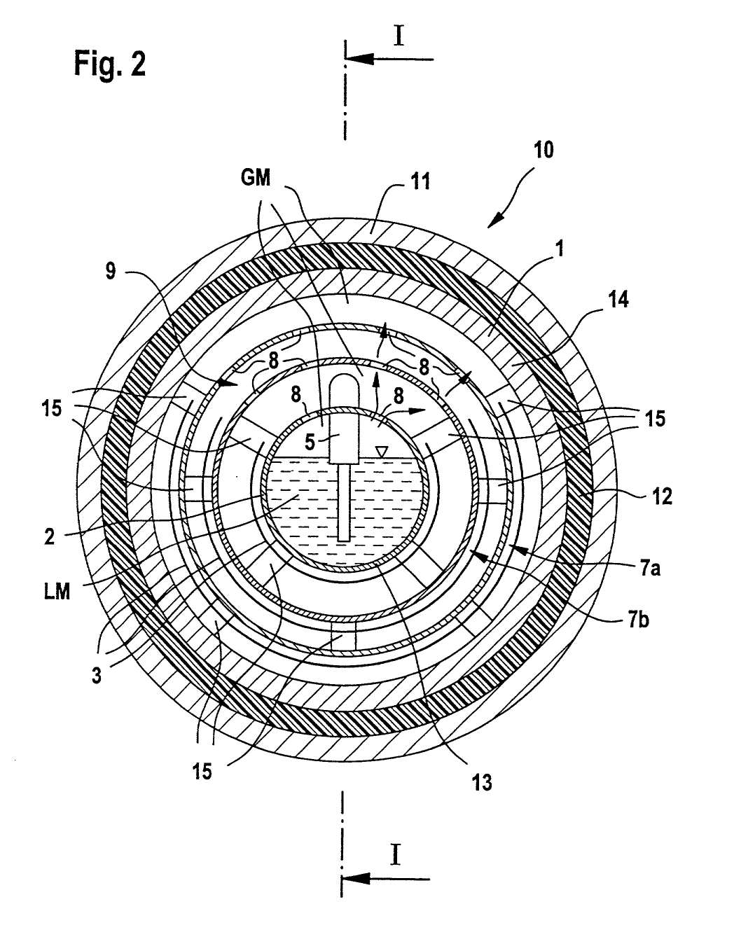

FIG. 2 shows a cross sectional view of the tank 10 according to FIG. 1, shortly after fueling liquid medium LM into the second vessel 2. The same elements as already described in FIG. 1 are referred to with the same reference numbers.

The liquid medium LM is contained in the second vessel 2. Mountings 15 are located between the first and second vessel walls 13, 14 and between the thermal screen layers 7a, 7b. Gaseous medium GM evaporates from the liquid medium reservoir out of the vessel 2 through the perforation holes 8 into the vessel 1. For hydrogen the volume of the liquid medium LM is only about 1/1000 of the volume of the medium GM in the gas phase at ambient pressure.

Vehicles with those storage tanks 10 will be able to refuel at any filling station providing high pressure or liquid hydrogen. Cheaper and simpler filling stations can be used if a high-pressure tank system vehicle is fueled with liquid hydrogen.

Advantageously the filling of a tank for high-pressure medium storage can be made faster as liquid medium lower pressure is required for refueling a tank.

The storage tank according to the invention allows the possibility to avoid high pressure equipment to fill high pressure hydrogen tanks and saves costs and increases safety of such systems.

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the appended claims and equivalents thereof.

What is claimed:

1. A storage tank for a medium with a first volume provided for containing a gaseous phase of said medium and a second volume provided for containing a liquid phase of said medium, a heater for said medium, an inlet and an outlet for said medium,
wherein the first volume is inside a first vessel and the second volume is inside a second vessel,
wherein said second vessel is in fluid connection with said first vessel via at least one fluid conduit, and
wherein said second vessel is surrounded by said first vessel.

2. A storage tank according to claim 1,
wherein a first inlet and outlet are provided for filling or discharging said gaseous medium into or out of said first vessel and a second inlet and outlet are provided for filling or discharging said liquid medium into or out of said second vessel.

3. A storage tank according to claim 1,
wherein said at least one fluid conduit is located in a region on top of said second vessel.

4. A storage tank according to claim 1,
wherein at least one thermal screen is arranged at least partially between said first and said second vessel.

5. A storage tank according to claim 4,
wherein said at least one screen comprises multiple shields, and
wherein a multiplicity of perforation holes is provided in each shield as said fluid conduit.

6. A storage tank according to claim 5,
wherein said perforation holes in neighboring shields are arranged in a staggered way so that said screen means are optically tight.

7. A storage tank according to claim 1,
wherein said inlet and outlet for the liquid medium comprises concentrically arranged conduits.

8. A storage tank according to claim 1,
wherein said inlet and outlet for said liquid medium and said inlet and outlet for said gaseous medium are arranged at the same side of said storage tank.

9. A storage tank according to claim 1,
wherein said outer wall of second vessel and/or at least one screen are in thermal contact to said heating.

10. A storage tank according to claim 1,
wherein additional heating means are arranged around said outer wall of said first vessel.

11. A storage tank according to claim 1,
wherein said outer wall of said first vessel is surrounded by reinforcement material.

12. A storage tank according to claim 1,
wherein said second vessel is made of steel and/or aluminum.

13. A storage tank according to claim 4,
wherein said screen means comprise reflective material.

14. A storage tank according to claim 1,
wherein the volume provided for containing said gaseous medium in said first vessel and in said second vessel is at least equal to the volume of said medium necessary to contain the completely evaporated liquid medium at a working pressure of said gaseous medium.

15. A storage tank according to claim 1,
wherein said working pressure of said gaseous medium is at least 100 bar.

16. Vehicle with a combustion engine, comprising a storage tank to store combustion fuel,
wherein said storage tank is for a combustion fuel medium with a first volume provided for containing a gaseous phase of said medium and a second volume provided for containing a liquid phase of said medium, a heater for said medium, and an inlet and an outlet for said medium,
wherein the first volume is inside a first vessel and the second volume is inside a second vessel, wherein said second vessel is in fluid connection with said first vessel via at least one fluid conduit, and
wherein said second vessel is surrounded by said first vessel.

17. Vehicle with a fuel cell system comprising a storage tank to store a fuel medium for the fuel cell, said storage tank having a first volume provided for containing a gaseous phase of said medium and a second volume provided for containing a liquid phase of said medium, a heater for said medium, an inlet and an outlet for said medium,
wherein the first volume is inside a first vessel and the second volume is inside a second vessel, wherein said second vessel is in fluid connection with said first vessel via at least one fluid conduit, and
wherein said second vessel is surrounded by said first vessel.

18. A storage tank for a medium comprising:
a first volume operable to contain a gaseous phase of said medium,
a second volume operable to contain a liquid phase of said medium,
a heater for heating said medium,
an inlet and an outlet for said medium,
wherein the first volume is inside a first vessel and the second volume is inside a second vessel, wherein said second vessel is in fluid connection with said first vessel via at least one fluid conduit, and
wherein said second vessel is surrounded by said first vessel.

19. A storage tank according to claim 18, p1 wherein said medium is hydrogen.

20. A storage tank according to claim 18,
wherein thermal screens are disposed between the first and second vessel, and
wherein at least one fluid conduit includes a plurality of perforations in said thermal screens.
